(19) United States
(12) Patent Application Publication
(10) Pub. No.: US 2013/0004548 A1
(43) Pub. Date: Jan. 3, 2013

(54) MEDICAL DEVICES HAVING PHARMACOLOGICAL ACTIVE AGENT RELEASING MATERIAL

(76) Inventors: Bjoern Klocke, Zuerich (CH); Laura Sager, Zuerich (CH); Mathias Gratz, Erlangen (DE)

(21) Appl. No.: 13/534,114

(22) Filed: Jun. 27, 2012

Related U.S. Application Data
(60) Provisional application No. 61/501,794, filed on Jun. 28, 2011.

(51) Int. Cl. A61K 33/06; A61K 33/08; A61F 2/82; A61P 9/10; A61F 2/84; A61K 9/00; A61P 9/00

(52) U.S. Cl. 424/400; 424/682; 424/692; 623/1.11; 623/1.2

(57) ABSTRACT

An embodiment of the present invention relates to a therapeutically active composition for the treatment of arteriosclerosis, and a pharmacological active agent-releasing medical device, the efficiency of which is increased by combination with a compound that releases alkaline-earth metal ions.

This application claims the benefit of U.S. application Ser. No. 61/501,794, filed Jun. 28, 2011, entitled Increase in Efficiency of the Therapeutic Effect of Pharmacological Active Agent Releasing Medical Devices, which is incorporated herein in its entirety.

### TECHNICAL FIELD

Some embodiments of the present invention relate to a therapeutically active composition for the treatment of arteriosclerosis, and a pharmacological active agent-releasing medical device comprising said therapeutically active composition, wherein the efficiency of the therapeutic effect of said active agents is increased by combining a compound that releases alkaline-earth metal ions in an aqueous environment with a pharmacological active agent.

### BACKGROUND

Cardiovascular diseases are one of the most common causes of death in the developed world, and coronary diseases are of primary significance. To treat these diseases, vascular prostheses, such as balloons or stents, are inserted intravascularly into the affected blood vessel of a patient and are implanted therein, if necessary, to expand it and hold it open.

However, the use of stents or other medical devices in some circumstances can initiate a cascade of microbiological processes which promote e.g. inflammation of the treated hollow organ, or a necrotic change, and which can result in a gradual closure of the stent, for instance, via the formation of plaques. In the worst case, this change in the hollow organ can result in restenosis or even closure of the hollow organ.

It is desirable to avoid the inflammation-promoting effect of medical devices to the greatest extent possible in the future, since the result is diminished efficacy of the medical device and the possibility of further damage to the organism being treated.

### SUMMARY

The problem addressed by at least some embodiments of the present invention is therefore that of providing a therapeutically active composition and a medical device which comprises said therapeutically active composition which, by combining a compound that releases alkaline-earth metal ions in an aqueous environment with a pharmacological active agent, increases the efficiency of the therapeutic effect of said active agents in terms of influencing the vitality, viability, proliferation, migration, and/or differentiation of cells of the vascular wall and adjacent tissue.

The problem is solved according to the invention by the features of the independent claims. Favorable embodiments and advantages of the invention will become apparent from the further claims and the description.

According to one aspect of the present invention, a therapeutically active composition which is composed of or contains at least one pharmacological active agent and at least one compound that releases magnesium ions in an aqueous environment is provided.

Another aspect of the present invention is a medical device which comprises a therapeutically active composition which is composed of or contains at least one pharmacological active agent and at least one compound that releases magnesium ions in an aqueous environment.

Another aspect of the present invention relates to the use of the therapeutically active composition according to the invention to manufacture a medical device.

Yet another aspect of the present invention relates to the use of the therapeutically active composition according to the invention for the prevention or therapy of a restenosis or an impairment of a vascular lumen in a section of a vessel.

### DETAILED DESCRIPTION

To prevent the risk factors of restenosis, a large number of coatings for balloons and stents were developed, which are intended to offer increased hemocompatibility. Anticoagulant, antimicrobial, anti-inflammatory, and antiproliferative agents have been used individually or in combination in the coating of balloons and stents. These substances are intended to be released from the coating material of the balloon or stent in such a way that they prevent inflammation of the surrounding tissue, excessive growth of smooth muscle cells, or blood clumping.

Paclitaxel, and derivatives thereof, is an active agent for the prevention of restenosis that is highly promising. This biocompatible substance is suited in particular for therapeutic use to diminish excessive cell proliferation in the vascular wall after balloon dilatation or stent implantation, since it is quickly absorbed into the vascular wall due to its lipophilic character, and is effective for a long time.

However, Paclitaxel in some circumstances can have a very disadvantageous effect on the expansion of blood vessels (vasodilatation), due to the reduced availability of nitrogen monooxide (NO), and on the healing properties of the irritated vascular wall, due to the inhibition of neointimal growth. These effects result in a clearly increased risk of thrombosis.

Surprisingly, it has been discovered that the therapeutically active composition according to the invention, via combination of a compound that releases magnesium ions in an aqueous environment with a pharmaceutical agent, with an effect on vitality, viability, proliferation, migration, and/or differentiation of cells of the vascular wall, including immigrating immune cells, and/or adjacent tissue, clearly increases the therapeutic effect of said active agents due to cell type-specific effects of magnesium ions on aforementioned cell activities.

The therapeutic effect within the scope of the present invention is understood to mean the sum of the biological, chemical, and physiological processes that are induced by the action of the pharmacological active agent.

The at least one pharmacological active agent is selected from taxanes, including, but not limited to, paclitaxel and derivatives thereof; sirolimus and derivatives thereof, including, but not limited to, biolimus A9, everolimus, zotarolimus, myolimus, novolimus, temsirolimus; mTOR inhibitors and substances from the group of epothilones or derivatives thereof.

The pharmacological active agents can be present in the therapeutically active composition individually or in combination, in the same concentration or in different concentrations. The at least one pharmacological active agent is present in the composition according to the invention preferably in a concentration of 0.0001-75% by weight, particularly preferably 0.001-25% by weight. According to the invention, the pharmacological active agent can also be present in the composition as a prodrug. A prodrug is understood to be a pharmacologically inactive or less active compound that must be in the organism to be converted into the particular active pharmacological active agent, as is the case with isotaxel, for instance, which is converted to paclitaxel once in the organism.

Suitable compounds that release magnesium ions in an aqueous environment include, but are not limited to, magnesium oxide, magnesium hydroxide, magnesium carbonate, magnesium aspartate and derivatives thereof, such as magnesium bi-(hydrogen-DL-aspartate), magnesium-L-diglutamate, magnesium hydrogen phosphate, magnesium citrate, magnesium sulphate, magnesium acetate, magnesium chloride, magnesium fumarate, magnesium gluconate, magnesium glycinate, magnesium lactate, magnesium salicylate, and magnesium stearate. In one embodiment, the compound that releases magnesium ions in an aqueous environment is selected from magnesium hydroxide and magnesium sulphate. The compounds which release magnesium in an aqueous environment can be present in the therapeutically active composition individually or in combination, in the same concentration or in different concentrations. The at least one compound that releases magnesium ions is present in the composition according to the invention preferably in a concentration of 25-99.9999% by weight, or 75-99.9990% by weight.

Among the advantages of the use of a compound that releases magnesium ions in an aqueous environment is the excellent biocompatibility. Compounds that release magnesium ions in an aqueous environment, such as magnesium salts, are among the substances that are essential to human nutrition and are indispensible to the organism. Magnesium must be supplied to the body via food or food supplements, to ensure that a large number of biological, chemical, and physiological processes can take place without limitation, since magnesium ions are required for the catalytic activity of many essential enzymes, among other things.

In one embodiment, the therapeutically active composition contains paclitaxel or sirolimus as the pharmacological active agent, and magnesium hydroxide or magnesium sulphate as a compound that releases magnesium in an aqueous environment.

In another embodiment, the therapeutically active composition can also contain, as an option, additional additives. Suitable additional additives, include, but are not limited to, sorbitol, titanium oxide, sodium lauryl sulphate, sodium hydrogencarbonate, potassium hydrogencarbonate, glycerol and derivatives thereof, resins such as shellac, and contrast medium, such as iopromid (Ultravist®). The additional additives are capable of positively controlling the availability of the pharmaceutical composition according to the invention at the site of action, i.e. extending the period of availability, controlling the tissue distribution, or increasing the maximum available concentration, for example.

Furthermore, the composition can be present as a solution containing the pharmacological active agent and the compound that releases magnesium in an aqueous environment, and other additives as an option. Suitable solvents include, but are not limited to, alcohols, such as ethanol; and acetone and butyryl trihexyl citrate.

Another aspect of the present invention relates to a medical device comprising a therapeutically active composition which is composed of or contains at least one pharmacological active agent and at least one compound that releases magnesium ions in an aqueous environment. In one embodiment, paclitaxel or sirolimus is used as the pharmacological active agent, and magnesium hydroxide or magnesium sulphate is used as the compound that releases magnesium in an aqueous environment.

In some embodiments, the therapeutically active composition covers the entire the medical device, while in others, it covers only part of the device.

According to the invention, a coating refers to the application, at least in sections, of the components on the base body of the medical device. In some embodiments, the coating covers the entire surface of the base body of the medical device. The layer thickness can be in the range of 1 μm to 100 μm, or 3 μm to 15 μm, although other thicknesses may be used, including those having a thickness of less than 1 μm and those having a thickness greater than 100 μm.

In some embodiments, the therapeutically active composition is present in a polymeric supporting matrix.

The polymeric supporting matrix can be composed of non-resorbable, permanent polymers, and/or resorbable, biodegradable polymers.

In some embodiments, the polymeric supporting matrix is composed of one or more polymers. Suitable polymers, include, but are not limited to, polyolefins, polyetherketones, polyethers, polyvinyl alcohols, polyvinyl halogenides, polyvinyl esters, polyacrylates, polyhalogen olefins, polyamides, polyamidimides, polysulfones, polyesters, polyurethanes, silicones, polyphosphazenes, polyphenylene, polymer foams (of styrolene and carbonates), polydioxanones, polyglycolides, polylactides, poly-c-caprolactone, ethyl vinyl acetate, polyethylene oxide, polyphosphorylcholine, polyhydroxybutyric acids, lipids, polysaccharides, proteins, polypeptides, and copolymers, blends, and derivatives of these compounds.

Examples of suitable polymers include, but are not limited to, polypropylene, polyethylene, polyisobutylene, polybutylene, polyetheretherketone, polyethylene glycol, polypropylene glycol, polyvinyl alcohols, polyvinyl chloride, polyvinyl fluoride, polyvinyl acetate, poly(ethyl acrylate), poly(methyl acrylate), polytetrafluorethylene, polychlorotrifluorethylene, PA 11, PA 12, PA 46, PA 66, polyamidimides, polyethersulfone, polyphenylsulfone, polycarbonates, polybutylene terephthalate, polyethylene terephthalate, elastanes, Pellethane, silicones, polyphosphazene, polyphenylene, polymer foams (of styrolene and carbonates), polydioxanones, polyglycolides, poly-L-, poly-D-, and poly-D,L-lactide, and poly-ε-caprolactone, ethyl-vinyl acetate, polyethylene oxide, polyphosphorylcholine, polyhydroxyvalerate, cholesterol, cholesterol ester, alginate, chitosan, levan, hyaluronic acid, uronides, heparin, dextrane, cellulose, fibrin, albumin, polypeptides and copolymers, blends, and derivatives of these compounds.

The polymeric supporting matrix can be selected in accordance with the desired elution rate and the individual characteristics of the various active agents that are used, and in accordance with the different rates of resorption and degradation at the site of action of the medical product.

The weight component of the polymeric supporting matrix according to the invention relative to the components of the coating forming the coating is generally at least 40%, or at least 70%, although other percentages can be used. The weight component of the at least one pharmacological active agent and the at least one compound that releases magnesium ions to the components of the coating forming the coating generally does not exceed 30%, or does not exceed 15%.

In some embodiments, the coating of the composition, which may be present in a polymeric supporting matrix, can be applied directly to the medical device. The processing can be performed using standard methods for the coating. Single-layered systems or multiple-layered systems (e.g. base coat layers, drug coat layers, or top coat layers) can be created. The coating can be applied directly to the base body of the device, or further layers can be provided therebetween to promote adhesion, for example.

In some embodiments, the at least one pharmacological active agent and the at least one compound that releases magnesium ions in an aqueous environment can be emitted from different polymeric supporting matrices.

Suitable medical devices are any types of medical products used, at least in part, for implantation in the body of a patient. Examples include, but are not limited to, tablets, capsules, stents, active agent pumps, cannulas, syringes, cardiac pacemakers, catheters, needle injection catheters, vascular transplants, balloons, organs, vessels, aortas, heart valves, tubes, organ replacement parts, fibers, hollow fibers, membranes, banked blood, blood containers, dialyzers, and sensors. In some embodiments, the medical device is a balloon or a stent.

A balloon according to the present invention is a balloon or balloon catheter known to a person skilled in the art, which is used in percutaneous transluminal angioplasty (PTA) or percutaneous transluminal coronary angioplasty (PTCA). These possibly multiple balloons are made of materials such as polyester, polyolefins, nylon, polyurethane, fluorpolymers, polyamides, polyethylene terephthalate (PET), polybutylene terephthalate (PBT), or polyether-polyurethanes and the copolymers and blends thereof. The balloon is preferably made of PEBAX.

A stent according to the present invention can be either biodegradable or permanent, and self-expanding or balloon-expandable.

Stents of a conventional design have a filigree support structure composed of metallic struts; the support structure is initially provided in an unexpanded state for insertion into the body, and is then widened into an expanded state at the application site. The stent can be coated before or after it is crimped onto a balloon.

The base body of the stent in some embodiments is composed of a metallic material composed of one or more metals or silicone. Suitable metals include, but are not limited to, iron, magnesium, nickel, tungsten, titanium, zirconium, niobium, tantalalum, zinc, platinum, or iridium. The base body can optionally include a second component composed of one or more metals. Suitable metals for the second component include, but are not limited to, lithium, sodium, potassium, calcium, manganese, iron, or tungsten. One example of a suitable second component is a zinc-calcium alloy.

In some embodiments, the base body is made of a memory effect material composed of one or more materials. Suitable memory effect materials include, but are not limited to, nickel-titanium alloys and copper-zinc-aluminum alloys. One example of a suitable memory effect material is Nitinol.

In some embodiments, the base body of the stent is composed of stainless steel. Suitable stainless steel includes, but is not limited to, a Cr-Ni-Fe steel, such as for example, the alloy 316L, or a Co-Cr steel. Furthermore, the base body of the stent can be composed, at least in part, of plastic and/or a ceramic.

Additionally, a passivating silicon carbide layer (SiC) can be provided on the base body of the stent composed of a metallic material, if desired. It is applied using a method known to a person skilled in the art and is located underneath the layer containing the composition according to the invention.

In some embodiments, the base body of the stent is composed of a biocorrodible metallic material, such as a biocorrodible alloy. Suitable biocorrodible alloys include, but are not limited to, magnesium, iron, and tungsten. An example of a suitable biocorrodible metallic material is a magnesium alloy.

A biocorrodible magnesium alloy is understood to be a metallic microstructure having magnesium as the main component. The main component is the alloy component that comprises the largest percentage by weight of the alloy. A percentage of the main component can be more than 50% by weight, or more than 70% by weight. The biocorrodible magnesium alloy can contain yttrium and other rare-earth metals, if desired, since an alloy of that type is characterized by its physical-chemical properties and high biocompatibility, in particular also the breakdown products thereof. One example of a suitable magnesium alloy is composed of 5.2-9.9% by weight of rare-earth metals, comprising 3.7-5.5% by weight of yttrium, and the rest <1% by weight thereof, wherein magnesium makes up the remainder of the alloy to reach 100% by weight. This magnesium alloy exhibits high biocompatibility, favorable processing properties, good mechanical characteristic values, and a corrosion behavior that is adequate for the intended uses. Another suitable magnesium alloy is composed of, the rest is <5% by weight thereof, and comprises e.g. 0.4-1.0% by weight of zirconium and 2.0-2.5% by weight of neodymium. In this case, the umbrella term “rare-earth metals” refers to scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70), and lutetium (71).

In some embodiments, the stent is composed of natural polymers such as collagen, chitin, chitosan, heparin. In some embodiments, the stent is made of degradable polymers, e.g. a polylactic acid such as PDLA, PLLA, PLGA, and P3HB, P4HB, or caprolactone and copolymers of the stated polymers.

The stent design is preferably adapted such that contact with the vessel wall is maximized. This promotes uniform elution of the pharmacological active agent.

Alternatively, in some embodiments, the coating of the composition can be present as cavity filling or as a component of a cavity filling. The medical device, such as the balloon or the stent, for example, comprises one or more cavities for this purpose. Cavities are located in the surface of the medical device, for instance, and can be created using methods known to a person skilled in the art depending on the device. In regard to the design of the cavity, a person skilled in the art can refer to the systems described in the prior art. In that particular case, the expression “cavity” refers to holes and recesses, for example. In some embodiments, the coating or cavity filling is contained within a cavity that defines a base body interior that is protected from exposure to the external environment by the base body, and that is only exposed to the external environment after a portion of the base body erodes or decays to thereby expose the interior cavity.

In some embodiments, the medical device, such as a balloon or a biodegradable or permanent, self-expandable or balloon-expandable stent, for example, comprises a therapeutically active composition that contains the pharmacological active agent, the compound that releases magnesium in an aqueous environment, and, optionally, other additives as defined above. In some embodiments, the medical device comprises a therapeutically active composition that contains paclitaxel or sirolimus as the pharmacological active agent, and magnesium hydroxide or magnesium sulphate as a compound that releases magnesium in an aqueous environment, and, optionally, other additives. The pharmacological active agent paclitaxel or sirolimus, and/or the compound that releases magnesium in an aqueous environment, magnesium hydroxide or magnesium sulphate, and/or the optional further additives can be present in the same polymeric supporting matrix or in different polymeric supporting matrices.

In some embodiments, the therapeutically active composition contains paclitaxel or sirolimus in quantities of 5 to 30 mg, or 15 to 25 mg, and magnesium sulphate or magnesium hydroxide in quantities of 50 to 900 mg, or 250 to 600 mg. In some embodiments, poly-L-lactide, poly-DL-lactide, or PGLA is used as the polymeric supporting matrix.

Furthermore, the composition can be present as a solution containing the pharmacological active agent and the compound that releases magnesium in an aqueous environment, and other additives as an option, and can then be coated onto the medical device. The particular components can be present in the same polymeric supporting matrix or in different polymeric supporting matrices. Suitable solvents include, but are not limited to, alcohols, such as ethanol; and acetone.

Magnesium ions have an intrinsic effect on the proliferation of smooth muscle cells: they reduce them more than the proliferation of endothelial cells. Due to the different mechanisms of action of paclitaxel or sirolimus in combination with a compound that releases magnesium in an aqueous environment, the combination undergoes an additive fortification.

Another aspect of the present invention is the use of a therapeutically active composition which is composed of or contains at least one pharmacological active agent and at least one compound that releases magnesium ions in an aqueous environment, for manufacturing a medical device.

Another aspect of the present invention is the use of a therapeutically active composition which is composed of or contains at least one pharmacological active agent and at least one compound that releases magnesium ions in an aqueous environment, for the prevention or therapy of a restenosis or an impairment of a vascular lumen in a section of a vessel.

### Example 1

Dissolve 30 mg paclitaxel in ethanol, then add macrogolglycerol ricinoleate. Add 900 mg magnesium sulphate and dissolve.

### Example 2

Dissolve 30 mg paclitaxel in ethanol, then add iopromid. Add 900 mg magnesium sulphate and dissolve.

### Example 3

Dissolve 30 mg paclitaxel in ethanol, then add macrogolglycerol ricinoleate. Add 900 mg magnesium hydroxide and dissolve.

### Example 4

Dissolve 30 mg paclitaxel in ethanol, then add iopromid. Add 900 mg magnesium hydroxide and dissolve.

### Example 5

Dissolve 30 mg paclitaxel in acetone containing PLGA, then add 900 mg magnesium hydroxide.

Medical devices such as tablets, capsules, stents, active agent pumps, cannulas, syringes, cardiac pacemakers, catheters, needle injection catheters, vascular transplants, balloons, organs, vessels, aortas, heart valves, tubes, organ replacement parts, fibers, hollow fibers, membranes, banked blood, blood containers, dialyzers, and sensors, in particular a balloon or a biodegradable or permanent, self-expandable or balloon-expandable stent can comprise the above-noted compositions according to the invention (examples 1 to 5).

For example, the composition according to the invention, per one of the examples 1 to 4, can be applied systemically using a syringe or a cannula, over a period of 30 minutes to three hours, or for one day or several days, e.g. 90 days.

Furthermore, the composition according to the invention, per one of the examples 1 to 5, can be applied to a biodegradable or permanent, self-expandable or balloon-expandable stent. Methods known to a person skilled in the art can be used to apply the composition according to the invention to the surface of the stent, and possibly to dry same. For instance, the coating can be applied by immersion or spraying onto the surface, and can be dried at 40° C. under vacuum. The surface of the stent can contain a concentration of paclitaxel and magnesium ion-releasing compound of 2.5 to 10.0 μg/mm² stent surface.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A therapeutically active composition comprising at least one pharmacological active agent and at least one compound that releases magnesium ions in an aqueous environment.

2. The therapeutically active composition according to claim 1, wherein the at least one pharmacological active agent is selected from taxanes, paclitaxel and derivatives thereof, sirolimus and derivatives thereof, biolimus A9, everolimus, zotarolimus, myolimus, novolimus, temsirolimus, mTOR inhibitors and substances from the group of epothilones or derivatives thereof.

3. The therapeutically active composition according to claim 1, wherein the at least one compound that releases magnesium ions in an aqueous environment is selected from magnesium oxide, magnesium hydroxide, magnesium carbonate, magnesium aspartate and derivatives thereof, magnesium-L-diglutamate, magnesium hydrogen phosphate, magnesium citrate, magnesium sulphate, magnesium acetate, magnesium chloride, magnesium fumarate, magnesium gluconate, magnesium glycinate, magnesium lactate, magnesium salicylate, and magnesium stearate.

4. The therapeutically active composition according to claim 1, wherein the pharmacological active agent is paclitaxel, and the compound that releases magnesium ions is magnesium hydroxide or magnesium sulphate.

5. The therapeutically active composition according to claim 1, wherein the pharmacological active agent is sirolimus, and the compound that releases magnesium ions is magnesium hydroxide or magnesium sulphate.

6. The therapeutically active composition according to claim 1, wherein the therapeutically active composition further comprises additional additives selected from sorbitol, titanium oxide, sodium lauryl sulphate, sodium hydrogencarbonate, potassium hydrogencarbonate, glycerol and derivatives thereof, and contrast medium.

7. A medical device comprising a therapeutically active composition comprising at least one pharmacological active agent and at least one compound that releases magnesium ions in an aqueous environment.

8. The medical device according to claim 7, wherein the therapeutically active composition covers the medical device entirely or covers part of the medical device.

9. The medical device according to claim 7 wherein the therapeutically active composition is present in a polymeric supporting matrix.

10. The medical device according to claim 9, wherein the at least one pharmacological active agent and the at least one compound that releases magnesium ions in an aqueous environment are present in different polymeric supporting matrices.

11. The medical device according to claim 7, wherein the device is selected from tablets, capsules, stents, active agent pumps, cannulas, syringes, cardiac pacemakers, catheters, needle injection catheters, vascular transplants, balloons, organs, vessels, aortas, heart valves, tubes, organ replacement parts, fibers, hollow fibers, membranes, banked blood, blood containers, dialyzers, and sensors.

12. The medical device according to claim 7, wherein the device is a balloon or a stent.

13. The medical device according to claim 12, wherein the device is the stent and wherein the stent is biodegradable or permanent.

14. The medical device according to claim 12, wherein the device is the stent and wherein the stent is self-expandable or balloon-expandable.

15. The medical device according to claim 7, wherein the therapeutically active composition comprises paclitaxel as the pharmacological active agent, and contains magnesium hydroxide or magnesium sulphate as the compound that releases magnesium ions, and, optionally, other additives.

16. The medical device according to claim 15, wherein the pharmacological active agent, the compound that releases magnesium ions, and the optional other additives are present in one polymeric supporting matrix or in different polymeric supporting matrices.

17. The use of a therapeutically active composition comprising at least one pharmacological active agent and at least one compound that releases magnesium ions in an aqueous environment for the prevention or therapy of a restenosis or an impairment of a vascular lumen in a section of a vessel.

18. The medical device according to claim 7, wherein the at least one pharmacological active agent is selected from taxanes, paclitaxel and derivatives thereof; sirolimus and derivatives thereof, biolimus A9, everolimus, zotarolimus, myolimus, novolimus, temsirolimus; mTOR inhibitors and substances from the group of epothilones or derivatives thereof.

19. The medical device according to claim 7, wherein the at least one compound that releases magnesium ions in an aqueous environment is selected from magnesium oxide, magnesium hydroxide, magnesium carbonate, magnesium aspartate and derivatives thereof, magnesium-L-diglutamate, magnesium hydrogen phosphate, magnesium citrate, magnesium sulphate, magnesium acetate, magnesium chloride, magnesium fumarate, magnesium gluconate, magnesium glycinate, magnesium lactate, magnesium salicylate, and magnesium stearate. The medical device according to claim 7, wherein the pharmacological active agent is sirolimus, and the compound that releases magnesium ions is magnesium hydroxide or magnesium sulphate.
